(19) United States
(12) Patent Application Publication
(10) Pub. No.: US 2026/0345221 A1
(43) Pub. Date: Nov. 5, 2026

(54) THROMBECTOMY DEVICES INCLUDING ONE OR MORE HEATABLE CUTTERS

(71) Applicant: Bard Peripheral Vascular, Inc., Franklin Lakes, NJ (US)

(72) Inventors: Brandon Simmons, Tempe, AZ (US); Clint Solomon, Gilbert, AZ (US); Daniel Matthew Foldes, Scottsdale, AZ (US); Andrew William Hamidy, Tempe, AZ (US)

(21) Appl. No.: 19/196,218

(22) Filed: May 1, 2025

(51) Int. Cl. A61B 18/08; A61B 18/00

(52) U.S. Cl. CPC A61B 18/082; A61B 2018/00083; A61B 2018/00202; A61B 2018/00601; A61B 2018/00714

(57) ABSTRACT

In an example, a thrombectomy device includes a catheter including a proximal end region and a distal end region. The thrombectomy device further includes at least one heatable cutter attached to or extending from the distal end region. The heatable cutter is configured to cut a thrombus. The thrombectomy device is switchable between a deployment state (e.g., at least partially collapsed state) and an expanded state. A maximum lateral dimension of the heatable cutter measured from a longitudinal axis of the catheter is greater when the thrombectomy device is in the expanded state than the deployment state.

### BACKGROUND

A thrombectomy is a surgical procedure that includes removal of a blood clot from a blood vessel (e.g., artery or vein) to restore blood flow in the blood vessel. The thrombectomy is often performed using catheter-based therapies. Thrombectomy may be an alternative to or performed in conjunction with thrombolytic medications. Thrombectomy may be used to treat any condition involving a thrombus, such as cerebral ischemic strokes, myocardial infarction, and pulmonary embolism.

### SUMMARY

Embodiments disclosed herein related to thrombectomy devices, systems including the same, and methods of using the same. In an embodiment, a thrombectomy device is disclosed. The thrombectomy device includes a catheter including a proximal end region and a distal end region. The thrombectomy device also includes at least one heatable cutter extending from the distal end region. The at least one heatable cutter is configured to cut a thrombus. The thrombectomy device is switchable between a deployment state and an expanded state, wherein a maximum lateral dimension of the thrombectomy device is greater when the thrombectomy device and the at least one heatable cutter are in the expanded state than in the deployment state.

In an embodiment, a thrombectomy system is disclosed. The thrombectomy device includes a thrombectomy device. The thrombectomy device includes a catheter including a proximal end region and a distal end region. The thrombectomy device also includes at least one heatable cutter extending from the distal end region. The at least one heatable cutter is configured to cut a thrombus. The thrombectomy device is switchable between a deployment state and an expanded state, wherein a maximum lateral dimension of the thrombectomy device is greater when the thrombectomy device and the at least one heatable cutter are in the expanded state than in the deployment state. The thrombectomy system also includes a stimulus source configured to provide stimuli to the at least one heatable cutter. The stimulus is configured to heat the heatable cutter.

In an embodiment, a method of using a thrombectomy device is disclosed. The method includes providing a thrombectomy device. The thrombectomy device includes a catheter including a proximal end region and a distal end region. The thrombectomy device also includes at least one heatable cutter extending from the distal end region. The at least one heatable cutter is configured to cut a thrombus. The method also includes switching the thrombectomy device from a deployment state to an expanded state. A maximum lateral dimension of the thrombectomy device and the at least one heatable cutter are greater when the thrombectomy device is in the expanded state than in the deployment state.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

FIGS. 1A and 1B are isometric views of a thrombectomy device in a deployment state and an expanded state, respectively, according to an embodiment.

FIG. 2 is a cross-sectional view of a heatable cutter, according to an embodiment.

FIG. 3A is a cross-sectional view of a heatable cutter, according to an embodiment.

FIG. 3B is a cross-sectional view of a heatable cutter, according to an embodiment.

FIG. 3C is a cross-sectional view of a heatable cutter, according to an embodiment.

FIG. 4 is a side view of a heatable cutter in the deployment state that has one or more weakened structures, according to an embodiment.

FIG. 5 is a schematic side view of a thrombectomy system, according to an embodiment.

The drawings illustrate several embodiments of the present disclosure, wherein identical reference numerals refer to identical or similar elements or features in different views or embodiments shown in the drawings.

### DETAILED DESCRIPTION

Embodiments disclosed herein related to thrombectomy devices, systems including the same, and methods of using the same. In an example, a thrombectomy device includes a catheter including a proximal end region and a distal end region. The thrombectomy device further includes at least one heatable cutter attached to or extending from the distal end region. The heatable cutter is configured to cut a thrombus. The thrombectomy device is switchable between a deployment state (e.g., at least partially collapsed state) and an expanded state. A maximum lateral dimension of the heatable cutter measured from a longitudinal axis of the catheter is greater when the thrombectomy device is in the expanded state than the deployment state.

During use, at least the distal end region of the catheter is inserted into a vessel (e.g., artery or vein) of an individual. The catheter may be advanced in the vessel in a distal direction (i.e., the direction extending along a central longitudinal axis of the catheter from the proximal end region to the distal end region) until the distal end region is proximate to or extends through a thrombus. At least initially, the thrombectomy device is in the deployment state to facilitate insertion of the catheter into the vessel and advancement of the catheter in the vessel. With the distal end region positioned proximate to the thrombus, the thrombectomy device may be switched from the deployment state to the expanded state by increasing the maximum lateral dimension of the heatable cutter. For example, the maximum lateral dimension of the heatable cutter may be increased until the heatable cutter contacts or is otherwise immediately adjacent to the walls of the vessel. The heatable cutter may then be heated to a temperature that is greater than the temperature of the vessel, such as to a temperature that is about 90 °C to about 130 °C. The heatable cutter may then contact and move into the thrombus. The temperature of the heatable cutter allows the heatable cutter to more easily cut the thrombus and remove the thrombus from the vessel than if the heatable cutter exhibited a temperature that is similar to the temperature of the vessel. In an example, the heatable cutter may be rotated about the longitudinal axis of the catheter as the heatable cutter contacts and removes the thrombus from the vessel.

The thrombectomy devices disclosed herein are an improvement over conventional thrombectomy devices. For example, conventional thrombectomy devices often include cutters that are used to cut and remove a thrombus. The cutters of these conventional thrombectomy devices are not heated and rely on force and sharpened cutting edges thereof to cut and remove the thrombus. However, it may be difficult to form sharpened cutting edges on the cutters of these conventional thrombectomy device because of the relatively small size of the cutters and the material (e.g., nickel-titanium alloys) that typically form the cutters is difficult to machine and/or retain the sharpened cutting edge. Further, some harder thrombi may be difficult for the cutters of conventional thrombectomy devices to cut, especially since the cutters of conventional thrombectomy devices are typically flexible. These issues associated with conventional thrombectomy device may only allow the thrombectomy device to cut small pieces of the thrombus which increases the time required to remove the thrombus. The small cut pieces of the thrombus also increases the likelihood that the cut pieces of the thrombus are not captured by the conventional thrombectomy device and instead form an embolus.

The thrombectomy devices disclosed herein resolve at least some of these issues associated with conventional thrombectomy devices. For example, the thrombectomy devices disclosed herein include at least one heatable cutter. When heated, the heatable cutter facilitates cutting of the thrombus. For instance, the heatable cutter may soften, burn, or otherwise damage the thrombus, each of which facilitate cutting or otherwise removing the thrombus with the heatable cutter. The heatable cutter may cut a thrombus using less force than a conventional cutter even if the heatable cutter does not include a sharpened cutting edge. The heatable cutter may also cut larger pieces of the thrombus, thereby decreasing the thrombectomy procedure and decreasing the likelihood of forming an embolism. As such, the heatable cutter may, compared to conventional thrombectomy devices, improve efficiency of the thrombectomy procedure and reduce the time needed to perform the thrombectomy procedure while also reducing the likelihood of forming emboli.

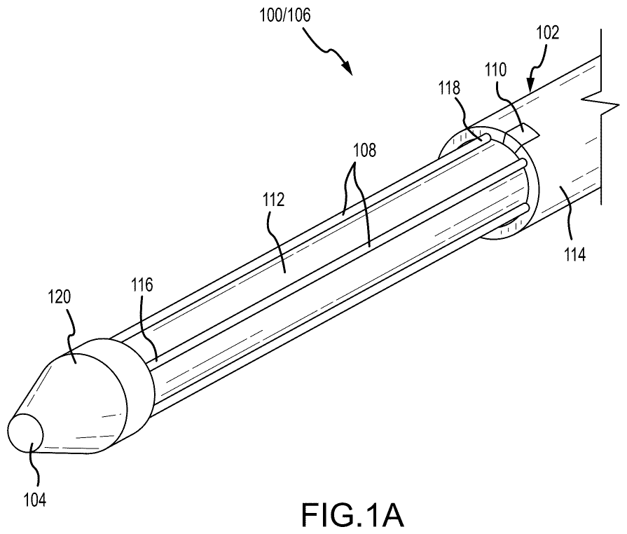

FIGS. 1A and 1B are isometric views of a thrombectomy device 100 in a deployment state and an expanded state, respectively, according to an embodiment. The thrombectomy device 100 includes a catheter 102 defining a hollow cavity 104. The catheter 102 includes a proximal end region (not shown) and a distal end region 106 (shown). The thrombectomy device 100 also includes a plurality of heatable cutters 108. The heatable cutters 108 are configured to be heated to a temperature that is greater than the temperature within a vessel (e.g., body temperature). The heatable cutters 108 are configured to vary the maximum lateral dimension (e.g., radius measured from a longitudinal axis of the catheter 102 or a diameter measured between two opposing heatable cutters 108). For example, the maximum lateral radius of the cutters may be greater when the thrombectomy device 100 is in the expanded state than when the thrombectomy device 100 is in the deployment state.

The catheter 102 provides a support body to support the other components (e.g., the heatable cutters 108, etc.) of the thrombectomy device 100. The catheter 102 also provides a means to move the components in a blood vessel by, for instance, pushing or pulling on the portions of the catheter 102 that are outside of the body. For example, the catheter 102 provides a means to move the other components of the thrombectomy device 100 through a vessel until the distal end region 106 is positioned proximate to or through a thrombus. The catheter 102 may also move the heatable cutters 108 into the thrombus and, optionally, rotate the heatable cutters 108 relative to the thrombus to cut at least a portion of the thrombus, for instance, from the walls of the vessel.

The catheter 102 is an elongated element that extends from a proximal end region (not shown) to a distal end region 106. During use, the proximal end region may be outside of a body. The distal end region 106 of the catheter 102 is the portion of the catheter 102 that the heatable cutters 108 are attached to and extend from. The catheter 102 may also define a hollow cavity 104 that is configured to receive a guidewire.

In an embodiment, the catheter 102 may be the same as or substantially to the catheters of conventional thrombectomy devices. In an embodiment, the catheter 102 may include at least one of a poly(ether-block-amide), a polyamide, a nylon, a polyimide, other thermoplastic elastomers, a reinforced polymer, any other suitable material, or combinations thereof. The catheter 102 may be distinct from a guidewire used to position the catheter 102 in an individual.

As previously discussed, the thrombectomy device 100 includes a plurality of heatable cutters 108. During use, the heatable cutters 108 may be exposed to one or more stimuli that are configured to heat the heatable cutters 108 to an elevated temperature (e.g., a temperature greater than an initial temperature of the heatable cutters 108 and/or a temperature that is greater than the temperature of the vessel). The stimuli may include, for example, electrical energy (e.g., a direct current and/or alternative current) flowing therethrough, vibrational energy (e.g., ultrasound waves or other sound waves), heat provided by thermal conductivity from a heatable element spaced from or embedded within the heatable cutter 108, or any other suitable stimuli. It is noted that the stimuli may be provided directly to the heatable cutters 108 (e.g., the electrical energy), may be provided from a portion of the catheter 102 that is distinct and separate from the heatable cutters 108 (e.g., a resistive heater disposed in the catheter 102 at or near the distal end region 106), or from a source outside of the body (e.g., ultrasound waves).

When exposed to the stimuli, the heatable cutter 108 may be heated to a temperature that is greater than the temperature of the vessel in which the distal end region 106 of the catheter 102 is dispose or at least a temperature that is greater than the temperature of the heatable cutters 108 before being exposed to the stimuli. In an example, the heatable cutters 108 may include an electrical resistive material such that flowing electrical energy through the electrical resistive material increases the temperature of the heatable cutter 108. The electrical energy may include, for instance, a radio frequency (“RF”) alternative current since it has been found that such electrical energy can quickly, efficiently, and controllably increase the temperature of the heatable cutters 108 to the desired temperature with low likelihood of heating the heatable cutter 108 non-negligibly above the desired temperature. In an example, the heatable cutters 108 may include a piezoelectric material that is configured to absorb the vibrational energy and either emit the absorbed energy as heat or supply electrical energy generated by the absorption of the vibrational energy to an electrical resistive material. In an example, the heatable cutter 108 includes a high thermal conductive material such that, when the heatable cutter 108 is exposed to thermal energy (e.g., from a heater disposed near, adjacent to, or within the heatable cutter 108), the thermal energy is able to quickly conduct into and heat the heatable cutter 108. Other stimuli that may be used to heat the heatable cutters 108 include, for example, electric fields (e.g., an RF electrical frequency field which, similar to RF alternative currents, can quickly, efficiently, and controllably heat heatable cutters 108 that include a dielectric material), any other suitable stimulus, or combinations of any of the stimuli disclosed herein.

The heatable cutters 108 may exhibit any suitable temperature while or after being exposed to the stimuli. In an embodiment, the heatable cutters 108 may be heated to a temperature (e.g., maximum temperature) of about 60 °C to about 150 °C, such as in ranges of about 60 °C to about 80 °C, about 70 °C to about 90 °C, about 80 °C to about 100 °C, about 90 °C to about 110 °C, about 100 °C to about 120 °C, about 110 °C to about 130 °C, about 120 °C to about 140 °C, or about 130 °C to about 150 °C. Heating the heatable cutters 108 facilitates cutting or otherwise removing the thrombus from the vessel. For example, contacting the heatable cutters 108 against the thrombus may cause the thrombus to at least one of soften, be damaged (e.g., burn), dry out, crack, or otherwise be easier to cut. As such, the temperature that the heatable cutters 108 exhibit may be selected to be sufficiently high enough to facilitate cutting or removal of the thrombus. It is noted that the temperature of the heatable cutters 108 that facilitates cutting or removal of the thrombus may vary for a variety of reasons, including the type or characteristics of the thrombus, the structure of the heatable cutter 108 (e.g., does the heatable cutter 108 include a sharpened cutting edge or not), and the flexibility of the heatable cutter 108. It is currently believed that heating the heatable cutters 108 to a temperature of about 90 °C or greater is sufficient for the heatable cutters 108 to cut most thrombi.

That said, heating the heatable cutters 108 may cause the heatable cutters 108 to damage the vessel or any fluids flowing in the vessel (e.g., blood). For example, heating the heatable cutters 108 to a temperature greater than 100 °C may cause any water within the vessel to boil. As such, in some embodiments, the temperature of the heatable cutters 108 may be selected to be less than 100 °C. That said, certain techniques may be used to mitigate damage to the vessel or the fluids flowing through the vessel. For example, the heatable cutters 108 may be moved when heated, for example, by rotating the distal end region 106 of the catheter 102, moving the distal end region 106 proximally (i.e., towards an incision through the distal end region 106 was inserted into a body of an individual), or moving the distal end region 106 distally (i.e., away from the incision). Such movement of the distal end region 106 minimizes the time that the heatable cutters 108 are in contact with any portion of the vessel or any fluid within the vessel which may minimize thermal energy that is transferred from the heatable cutters 108 to the vessel or the fluid. Further, heating the heatable cutters 108 facilitates cutting or otherwise removing the thrombus from the vessel thereby limiting the time that the heatable cutters 108 are within the vessel compared to conventional thrombectomy devices which, in turn, minimizes any damage to the vessel or the fluid in the vessel caused by the heatable cutters 108. In an example, the heatable cutters 108 may exhibit a maximum lateral dimension (e.g., a diameter of a periphery of the heatable cutters 108 rotated about the longitudinal axis that is double the maximum radius) that is less than the diameter of the lumen defined by the walls of the vessel thereby minimizing contact with the walls of the vessel. It is currently believed that heating the heatable cutters 108 to a temperature of about 130 °C or less is sufficient to minimize damage to the vessel and the fluid within the vessel, for example, when using one or more of the techniques disclosed above.

In a particular embodiment, the thrombectomy device 100 may be used to cut a thrombus from within a stent disposed in a vessel. For example, stents often cause the formation of a thrombus therein over prolonged periods of time. The thrombectomy device 100 may be isolated from the vessel by the stent. As such, the temperature of the heatable cutters 108 is less likely to damage the vessel. It is noted that the temperature of the heatable cutters 108 is unlikely to damage the stent.

In an embodiment, the thrombectomy device 100 includes one or more sensors 110 that are configured to detect the temperature of the heatable cutters 108. For example, the thrombectomy device 100 may include one or more sensors 110 adjacent to, contacting, disposed on, or disposed in one or more of the heatable cutters 108. The sensors 110 may include any suitable temperature sensor, such as a bolometer (e.g., microbolometer), a bimetallic strip, a heat flux sensor, a resistance thermometer, or any other suitable sensor. It is noted that the thrombectomy device 100 may include sensors 110 other than temperature sensors, such as a current or voltage sensor configured to detect the properties of the electrical energy provided to the heatable cutters 108.

In an embodiment, the temperature of the heatable cutters 108 may be controlled in response to the temperature detected by the sensors 150. In an example, stimuli may be provided to the heatable cutters 108 when the temperature is below a selected or desired temperature, for instance, during initial heating of the heatable cutters 108 or if the temperature of the heatable cutters 108 drops below the selected or desired temperature during use. In an example, a relatively low intensity stimuli may be applied to the heatable cutters 108 during use to maintain the heatable cutters 108 at or near the selected or desired temperature. In an example, the stimuli may not be provided to the heatable cutters 108 when the temperature of the heatable sensors 150 is above the selected or desired temperature.

In an embodiment, the temperature of the heatable cutters 108 may be determined during use. In an example, a user of the thrombectomy device 100 (e.g., surgeon) may determine that the temperature of the heatable cutters 108 is not sufficiently high enough because the heatable cutters 108 are not cutting the thrombus in a desired manner. In such an example, the user may cause the stimuli applied to the heatable cutters 108 to be increased (e.g., increase the intensity of the stimuli) thereby causing the temperature of the heatable cutters 108 to be increased. The user may cause the stimuli to be increased until the heatable cutters 108 cut the thrombus in a desired manner. In an example, the user of the thrombectomy device 100 may determine that the temperature of the heatable cutters 108 is too high, for instance, by observing damage to the vessel during use (e.g., using ultrasound imaging) or due to examining the temperature of the heatable cutters 108 detected by the sensors 150. In such an example, the user may cause the stimuli applied to the heatable cutters 108 to be decreased (e.g., decrease the intensity of the stimuli) or stop the stimuli from being applied to the heatable cutters 108.

The heatable cutter 108 may be formed of any suitable material. For example, the heatable cutter 108 may include at least one nickel-titanium alloy (e.g., nitinol®), stainless steel, nickel-chromium alloy (e.g., nichrome®), or another a resistive heatable material, a biocompatible material that is good thermal conductor, a biocompatible insulator (e.g., biocompatible polymer), a dielectric material, or any other suitable material. In an example, the heatable cutters 108 may include a material that is heated when exposed to the stimuli (e.g., electrical energy, ultrasound energy, etc.). As such, the material of the heatable cutter 108 may be selected based on the stimuli that is configured to heat the heatable cutter 108. In an example, the heatable cutters 108 may include at least one first material that is heated when exposed to the stimuli and at least one second material that is not heated when exposed to the stimuli.

In a particular embodiment, the heatable cutter 108 may include at least one nickel-titanium alloy (e.g., nitinol). Nickel-titanium alloys are often used to form the components of cutters in conventional thrombectomy devices since nickel-titanium alloys may exhibit at least one of a shape memory effect and/or superelasticity which may facilitate expanding and collapsing of the cutters. However, it is difficult to form a sharpened cutting edge in nickel-titanium alloys and, even if a sharpened cutting edge is formed in the nickel-titanium alloy, the nickel-titanium alloy may be unable to maintain the sharpened cutting edge during use. Further, the superelasticity of the nickel-titanium alloy may cause the cutters of conventional thrombectomy devices to deflect rather than cut a thrombus from a vessel, especially if the thrombus is relatively hard or the cutting edge of the cutter has dulled. The heatable cutters 108 disclosed herein resolve at least some of these issues associated with using nickel-titanium alloys in conventional thrombectomy devices even when the heatable cutters 108 include at least one nickel-titanium alloy. In an example, as will be discussed in more detail below, the heatable cutters 108 disclosed herein may not have a sharpened cutting edge since the heatable cutters 108, when heated, may be able to cut the thrombus from the vessel. In such an example, the heatable cutters 108 do not need to have the sharpened cutting edge formed therein. In an example, the heatable cutters 108, when heated, may reduce deflection of the cutters during use even when the heatable cutters 108 include a non-sharpened cutting edge, a dulled sharpened cutting edge, and/or the heatable cutters 108 are cutting or otherwise removing a hard thrombus from the vessel.

The thrombectomy device 100 may include any suitable number of heatable cutters 108. For example, the thrombectomy device 100 may include 1, 2, 3, 4, 5, 6, 7, 8, 9, 10, 11, 12, or more than 12 heatable cutters 108. Generally, increasing the number of heatable cutters 108 may decrease the force needed to cut the thrombus. However, increasing the number of heatable cutters 108 decreases the size of the cut pieces of the thrombus. The decreased size of the cut pieces of the thrombus make increase the likelihood that the thrombectomy device 100 or another device fails to remove the cut pieces of thrombus and the cut pieces of thrombus form emboli. As such, the number of heatable cutters 108 may be selected to ensure that the size of the cut pieces of thrombus are likely to be captured and removed from the vessel using the thrombectomy device 100 or another device. Also, increasing the number of heatable cutters 108 may require more stimuli to be heated to the desired temperature. In most applications, 4 to 8 heatable cutters 108 may be able to efficiently cut the thrombus with relatively low risk of forming emboli or requiring excessive stimuli to be heated.

The thrombectomy device 100 is configured to controllably switch from a deployment state to an expanded state. In the deployment state, the heatable cutters 108 are positioned adjacent to the catheter 102, are relatively straight, or otherwise exhibit a maximum lateral dimension or radius that is comparable to the catheter 102 (e.g., greater than the maximum lateral dimension or radius of the catheter 102 by, at most, about 2 times, or more particularly 1.5 times). The deployment state of the thrombectomy device 100 facilitates insertion of the distal end region 106 of the catheter 102 (including the heatable cutters 108) into the individual and movement of the distal end region 106 through the vessel to a desired location (e.g., the distal end region 106 is positioned proximal to or through a thrombus). Once the thrombectomy device 100 is positioned in the desired location, the thrombectomy device 100 may switch to the expanded state by causing the maximum lateral dimension of the heatable cutters 108 to increase.

In an example, the maximum lateral dimension of the thrombectomy device 100 when the heatable cutters 108 are in the expanded state may be substantially similar to an inner lateral dimension of the vessel. It is noted that the heatable cutters 108 may form the maximum lateral dimension of the thrombectomy device 100 when the heatable cutters 108 are in the expanded state. In an example, the maximum lateral dimension of the thrombectomy device 100 when the heatable cutters 108 in the expanded state may be less than the inner lateral dimension of the vessel and then increased (e.g., incrementally or continuously increase) during use until the maximum lateral dimension is similar to the inner dimension of the vessel. For instance, the thrombectomy device 100 may first exhibit a first maximum lateral dimension that is less than the lateral dimension of the vessel. When the thrombectomy device 100 exhibits the first maximum lateral dimension, the heatable cutters 108 may be used to cut a first portion of the thrombus. After cutting or otherwise removing the first portion of the thrombus, the maximum lateral dimension of the thrombectomy device 100 may be increased to a second maximum lateral dimension (e.g., a maximum lateral dimension that is still smaller than or generally equal to the inner dimension of the vessel). When the thrombectomy device exhibits the second maximum lateral dimension, the heatable cutters 108 may cut a second portion of the thrombus. If needed, the maximum lateral dimension of the thrombectomy device 100 may be increased one or more additional times to remove one or more additional portions of the thrombus until all or substantially all of the thrombus is cut. It is noted that increasing the maximum lateral dimension of the thrombectomy device 100 may minimize damage to the vessel during use and may better accommodate variations in the lateral dimensions of the vessel.

The thrombectomy device 100 may switch from the deployment state to the expanded state using any suitable technique. In an embodiment, as shown, the catheter 102 includes an inner catheter 112 and an outer catheter 114. The outer catheter 114 may be positioned to receive the inner catheter 112 and the outer catheter 114 is configured to move relative to the inner catheter 112. The heatable cutters 108 may include a distal end 116 and a proximal end 118 opposite the distal end 116. The distal end 116 of the heatable cutters 108 may be attached to the inner catheter 112 (e.g., at or near the distalmost portion of the inner catheter 112, excluding the optional atraumatic tip 120) and the proximal end 118 may be attached to or near the distalmost end of the outer catheter 114. The movement of the outer catheter 114 relative to the inner catheter 112 may control the maximum lateral dimension of the heatable cutters 108. For example, moving the outer lumen 114 in a proximal direction relative to the inner catheter 112 (e.g., increasing the distance between the distalmost tips of the inner catheter 112 and the outer catheter 114) causes the heatable cutters 108 to straighten out and move towards the inner catheter 112 thereby switching the thrombectomy device 100 to the deployment state. Moving the outer catheter 114 in a distal direction relative to the inner catheter 112 (e.g., decreasing the distance between the distalmost tips of the inner catheter 112 and the outer catheter 114) causes the heatable cutters 108 to deflect outwardly and increase the maximum lateral dimension thereof thereby causing the thrombectomy device 100 to exhibit the expanded state.

The thrombectomy device 100 may switch from the deployment state to the expanded state using any other suitable technique. In an embodiment, the heatable cutters 108 may include a shape memory material, such as a nickel-titanium alloy. The shape memory material of the heatable cutter 108 may exhibit a remembered shape that corresponds to or is greater than the inner lateral dimension of the vessel. Before use, the shape memory material of the heatable cutter 108 may be deformed until the thrombectomy device 100 is in the deployment state. That is, the shape memory material of the heatable cutters 108 may be deformed to be at least one of positioned adjacent to the catheter 102, are relatively straight, or otherwise exhibits a maximum lateral dimension or radius that is comparable to the catheter 102. After the distal end region 106 of the catheter 102 is correctly positioned, the heatable cutters 108 may be heated to a temperature that is sufficient to cause the shape memory material to assume its remembered shape. For example, the body heat or the heat caused by heating the heatable cutters 108 with stimuli may be used to cause the shape memory material to assume the remembered shape. It is noted that, if the inner lateral dimensions of the vessel are less than the lateral dimension of the remembered shape, that the vessel may prevent the heatable cutters 108 from assuming the remembered shape.

In an embodiment, the thrombectomy device 100 may include an outer sheath (not shown) defining a passageway that is configured to receive the catheter 102. The outer sheath is also slidable relative to the catheter 102. When the outer sheath positioned over the heatable cutters 108, the heatable cutters 108 are forced into the passageway of the outer sheath which, in turn, causes the heatable cutters 108 to be positioned adjacent to the catheter 102, are relatively straight, or otherwise exhibits a maximum lateral dimension or radius that is comparable to the catheter 102. In other words, positioning the outer sheath such that the heatable cutters 108 are at least partially within the passageway causes the thrombectomy device 100 to be in the deployment state. Sliding the outer sheath such that the heatable cutters 108 are no longer disposed in the inner passageway allows the heatable cutters 108 to expand. The heatable cutters 108 may be formed of a superelastic material (e.g., a nickel-titanium alloy), the thrombectomy device 100 may include springs which allows the heatable cutters 108, or the thrombectomy device 100 includes other materials or structures that allow the heatable cutters 108 to expand when the heatable cutters 108 are no longer disposed in the passageway. Moving the outer sheath back over the heatable cutters 108 may switch the thrombectomy device 100 back to the deployment state which may facilitate extraction of the distal end region 106 of the catheter 102 from the vessel. It is noted that the outer sheath may be used when the heatable cutters 108 include a shape memory material such that moving the outer sheath over the heatable cutters 108 (e.g., moving the heatable cutters 108 into the inner passageway) decreases the maximum lateral dimension of the heatable cutters 108.

The heatable cutters 108 may exhibit any suitable maximum lateral dimension when the thrombectomy device 100 is in its most expanded state (i.e., when the heatable cutters 108 exhibit their largest maximum lateral dimension). For example, the heatable cutters 108 may exhibit a largest maximum lateral dimension of about 7.5 mm to about 25 mm, such as in ranges of about 7.5 mm to about 12.5 mm, about 10 mm to about 15 mm, about 12.5 mm to about 17.5 mm, about 15 mm to about 20 mm, about 17.5 mm to about 22.5 mm, or about 20 mm to about 25 mm. The largest maximum lateral dimension is generally selected to be generally equal to or slightly larger than the inner lateral dimension of the portion of the vessel including the thrombus to ensure that the heatable cutters 108 can remove all or substantially all of the thrombus from the vessel. It is noted that the largest maximum lateral dimension of the heatable cutters 108 of about 14 mm to about 16 mm is sufficient for use in most vessels.

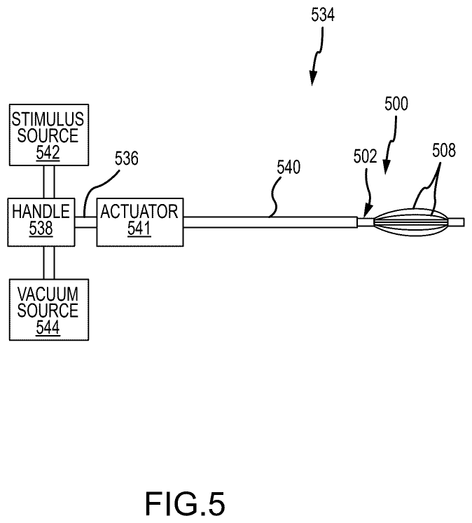

As previously discussed, the heatable cutters 108 may cut the thrombus from the vessel by rotating the heatable cutters 108 in the vessel while the thrombectomy device 100 is in the expanded state. The heatable cutters 108 may be rotated using any suitable technique. In an example, the heatable cutters 108 may rotate by rotating the entire thrombectomy device 100. In an example, the thrombectomy device 100 may include a handle (e.g., handle 538 of FIG. 5) and the handle includes an actuator. Manipulating the actuator may cause the distal end region 106 of the catheter 102, which includes the heatable cutters 108, to rotate. In some examples, rotating the heatable cutters 108 includes rotating the heatable cutters 108 in a 360° arc. In some examples, rotating the heatable cutters 108 includes rotating the heatable cutters 108 less than a 360° arc. In such examples, rotating the heatable cutters 108 may include rotating the heatable cutters 108 at least in an arc exhibiting, in degrees, an angle greater than [the number of heatable cutters 108]/360°. It is noted that rotating the heatable cutters 108 can include rotating the heatable cutters 108 in one circumferential direction or both circumferential directions (e.g., a back-and-forth rotation).

In an embodiment, the thrombectomy device 100 is configured to provide aspiration to the vessel at least concurrent with and/or after the heatable cutters 108 cut the thrombus. The aspiration may be used to suction the cut pieces of the thrombus from the vessel, thereby preventing the cut pieces of the thrombus from forming emboli. In an example, the aspiration may be provided to the vessel via the hollow cavity 104. In such an example, the cut pieces of the thrombus may be sucked into and removed from the vessel via the hollow cavity 104. In an example, the aspiration may be provided by one or more channels defined by the catheter 102 (not shown) that are distinct from the hollow cavity 104. In such an example, the aspiration may be provided to the vessel even if a guidewire or other medical device is positioned in the hollow cavity 104.

In an embodiment, the thrombectomy device 100 includes a capture bag (not shown). The capture bag may be a mesh bag or other porous material defining a plurality of gaps therein that allows blood or other bodily fluids to pass therethrough while substantially preventing the cut pieces of thrombus from passing therethrough. As such, the capture bag is configured to receive and hold the cut pieces of the thrombus (e.g., pieces of the thrombus that are not removed via the aspiration) thereby minimizing the risk of forming one or more emboli. It is noted that the capture bag may switch between a deployment state (e.g., the capture bag is disposed in a sheath) and an expanded state. The capture bag is generally positioned downstream of the selected material. In an example, as shown, the capture bag is attached to and extends from the heatable cutters 108 when the heatable cutters 108 are positioned downstream of the selected material. In an example, the capture bag may include a nickel-titanium alloy that is configured to expand when deployed from the central passageway. In an embodiment, the thrombectomy device 100 may include an embolic cap instead of or in addition to the capture bag.

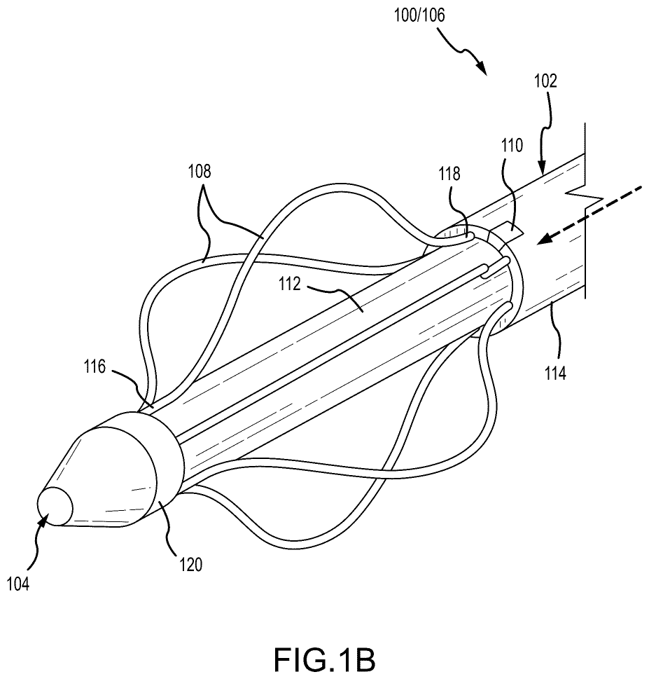

FIG. 2 is a cross-sectional view of a heatable cutter 208, according to an embodiment. The heatable cutter 208 includes two or more materials. For example, the heatable cutter 208 may include an inner (e.g., core) material 222 and an outer material 224 that at least partially surrounds the inner material 222. The outer material 224 may be, for example, a coating formed on the inner material 222 formed during any suitable technique (e.g., chemical vapor deposition, physical vapor deposition, dip coating, etc.). It is noted that the heatable cutter 208 may include one or more additional materials, such as an intermediate material disposed between the inner material 222 and the outer material 224.

Forming the heatable cutter 208 from two or more materials may facilitate at least one of the manufacturing or function of the heatable cutter 208. In an embodiment, the stimuli may include electrical energy that is flowed through the heatable cutter 208. In such an embodiment, the inner material 222 may include an electrical resistive heatable material (e.g., a nickel-chromium alloy) and the outer material 224 is an electrical insulating material (e.g., a biocompatible polymer) that prevents or at least inhibits the electrical energy flowing through the inner material 222 from shocking or otherwise flowing into the vessel or the fluid flowing in the vessel. In an embodiment, the inner material 222 or the outer material 224 may include a material exhibiting a thermal conductivity that is greater than the other of the inner material 222 or the outer material 224 which may cause the heatable cutter 208 to exhibit a more uniform temperature during use. In an embodiment, the inner material 222 may include a nickel-titanium alloy while the outer layer may include a material that may be easier to have a sharpened cutting edge formed therein, such as stainless steel. In an embodiment, the inner material 222 may include a non-biocompatible material and the outer material 224 may include a biocompatible material thereby allowing the heatable cutter 208 to be formed from additional materials.

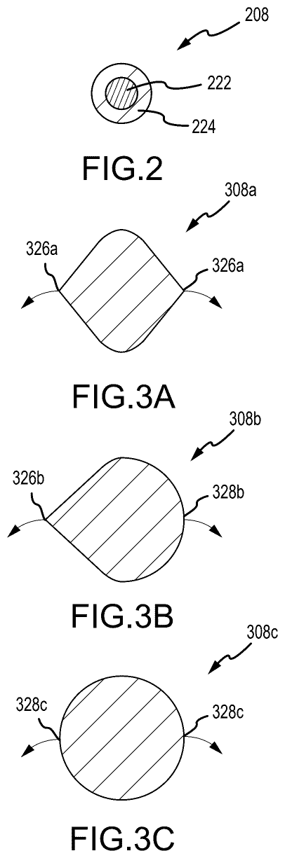

The heatable cutters disclosed herein may exhibit a variety of cutting edges 326a, as shown in FIGS. 3A-3C. FIG. 3A is a cross-sectional view of a heatable cutter 308a, according to an embodiment. The heatable cutter 308a includes two sharpened cutting edges 326a on opposing sides of the heatable cutter 308a. As used herein, a cutting edge is an edge of a heatable cutter that generally faces the direction of rotation (shown schematically with arrows). The sharpened cutting edges 326a of the heatable cutter 308a may facilitate cutting or otherwise removing the thrombus from the vessel compared to a substantially similar heatable cutter 308a that does not include the sharpened cutting edge. The two sharpened cutting edges 326a allows the heatable cutter 308a to cut the thrombus with a cutting edge regardless of the direction that the distal end region 306 of the catheter 302 is rotated within the vessel.

FIG. 3B is a cross-sectional view of a heatable cutter 308b, according to an embodiment. The heatable cutter 308b includes two cutting edges, a first sharpened cutting edge 326b and a second non-sharpened (e.g., rounded or circular) cutting edge 328b. The first sharpened cutting edge 326b is able to more easily cut the thrombus than the second, non-sharpened cutting edge 328b. However, as previously discussed, the heatable cutters 308b do not need a sharpened cutting edge to cut the thrombus from the vessel. The heatable cutter 308b may facilitate manufacturing of the heatable cutter 308b since only one sharpened cutting edge needs to be formed in the heatable cutter 308b. Also, the heatable cutter 308b allows the user of the thrombectomy device including the heatable cutter 308b to use either the first sharpened cutting edge 326b or the second non-sharpened cutting edge 328b as desired by changing the direction that the distal end region 306 of the catheter 302 is rotated. For example, the user may use the first sharpened cutting edge 326b when cutting a relatively hard thrombus and may use the second non-sharpened cutting edge 328b when penetration of the vessel walls is a concern or the first sharpened cutting edge 326b becomes entangled with a stent.

FIG. 3C is a cross-sectional view of a heatable cutter 308c, according to an embodiment. The heatable cutter 308c includes two, non-sharpened cutting edges 328c. As previously discussed, the heatable cutter 308c is able to cut most (if not all) types of thrombus even through the heatable cutter 308c does not include a sharpened cutting edge. That said, it is noted that the heatable cutter 308c may need to exhibit a higher temperature than a substantially similar heatable cutter 308c than includes a sharpened cutting edge.

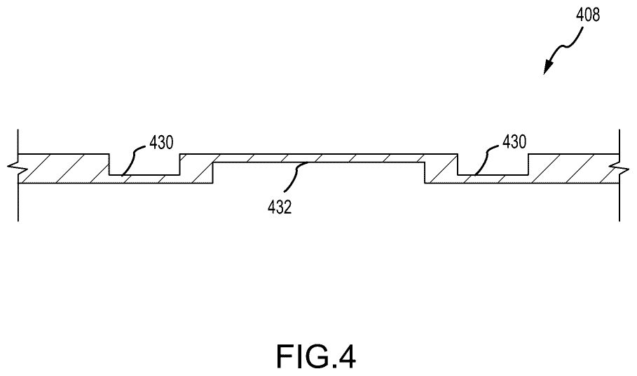

The heatable cutters disclosed herein may exhibit any suitable shape, such as a curve shape. The shape of the heatable cutters may be a preselected shape. The preselected shape of the heatable cutters may be selected to facilitate removal of a particular type of thrombus, operate within a particular vessel, or used to remove a thrombus from a particular stent. In an embodiment, the heatable cutters may be bent to exhibit the preselected shape. For instance, the heatable cutters may include a nickel-titanium alloy or other shape memory material and the preselected shape may be the “remembered” shape of the shape memory material. In an embodiment, the heatable cutters may include one or more selectively weakened portions that cause the heatable cutters to form the preselected shape. For example, FIG. 4 is a side view of a heatable cutter 408 in the deployment state that has one or more weakened structures, according to an embodiment. Except as otherwise disclosed herein, the heatable cutter 408 may be the same as or substantially similar to any of the heatable cutter 408s disclosed herein and may be used in any of the thrombectomy devices disclosed herein.

The heatable cutter 408 includes one or more thinned sections that form the weakened structures of the heatable cutter 408. The thinned sections of the heatable cutter 408 form portions of the heatable cutter 408 that are more likely to bend or at least exhibit larger bends than the non-thinned sections of the heatable cutter 408. In the illustrated embodiment, the heatable cutter 408 includes two outer thinned sections 430 and at least one inner thinned section 432 between the two outer thinned sections 430. The two outer thinned sections 430 and the inner thinned section 432 may be formed by one or more cutouts. The cutouts of the two outer thinned sections 430 may be formed on opposing sides of the heatable cutter 408 than the cutouts of the inner thinned section 432. Forming the cutouts on different sides of the heatable cutter 408 may control how each thinned sections curve when a compressive force is applied thereto. For instance, relative to the side of the heatable cutter 408 that includes the cutouts forming the two outer thinned sections 430, the two outer thinned sections 430 may form convex curves and the inner thinned section 432 may form a convex curve when a compressive force is applied to the heatable cutter 408 and the heatable cutter 408 buckles. Such a structure may allow the heatable cutter 408 to form the curved shape shown in FIG. 1B.

FIG. 5 is a schematic side view of a thrombectomy system 534, according to an embodiment. The system 534 includes a thrombectomy device 500 that is the same as or substantially similar to any of the thrombectomy devices disclosed herein. For example, the thrombectomy system 534 includes a catheter 502. The catheter 502 may include a proximal end region 536 and a distal end region 506. The proximal end region 536 may be attached to a handle and the distal end region 506 may include a plurality of heatable cutters 508. The handle 538 may be configured to move the catheter 502 when the catheter 502 is inserted into an individual. For example, moving the handle 538 in a distal direction may advance the catheter 502 into an individual and moving the handle 538 in a proximal direction may retract the catheter 502 from the individual. It is noted that the handle 538 may exhibit any structure known in the art and may move (e.g., proximally, distally, or rotate) the catheter 502 using any conventional technique.

The thrombectomy system 534 may also include a sheath 540 configured to switch the heatable cutters 508 between a deployment state and an expanded state. The sheath 540 is configured to slide along the catheter 502. Sliding the sheath 540 in a distal direction may cause the sheath 540 to receive the heatable cutters 508. Receiving the heatable cutters 508 into the sheath 540 may cause the heatable cutters 508 to be in the deployment state by forcing the maximum lateral dimension of the heatable cutters 508 to decrease until the maximum lateral dimension of the heatable cutters 508 is small enough to fit within the sheath 540. For example, the heatable cutters 508 are radially collapsible to a smaller diameter to be contained at least partially within the sheath 540 for insertion and retrieval. Sliding the sheath 540 in a proximal direction may cause the sheath 540 to expose the heatable cutters 508, thereby allowing the heatable cutters 508 to switch from the deployment state to the expanded state. Exposing the heatable cutters 508 allows the maximum lateral dimension of the heatable cutters 508 to increase. In an embodiment, the thrombectomy system 534 may include an actuator 541 that, when actuated, causes the sheath 540 to move in the distal direction or the proximal direction. The actuator 541 may be distinct (e.g., spaced) from the handle 538 (as shown) or form part of the handle 538. It is noted that, as previously discussed herein, the thrombectomy device 500 may be switched between the deployment and expanded states using techniques other than the sheath 540.

The thrombectomy system 534 may include a stimulus source 542. The stimulus source 542 is configured to apply a stimulus to the heatable cutters 508 that is selected to heat the heatable cutters 508 to an elevated temperature. The stimulus source 542 may be positioned at least partially outside of the thrombectomy device 500. The stimulus source 542 may include any stimulus source that is configured to provide any of the stimuli disclosed herein. In an example, the stimulus source 542 is an electrical energy source that is configured to provide electrical energy (e.g., a direct current or alternating current electrical energy) to the heatable cutters 508. In a particular example, the stimulus source 542 is configured to provide an alternating electrical current in the radio frequency ranges. In an example, the stimulus source 542 may include an ultrasound source, another vibrating source, an electrical energy source configured to provide electrical energy to a heater, an electrical field source, or any other suitable stimulus source.

In an embodiment, the thrombectomy system 534 may include a vacuum source 544 configured to provide aspiration to the vascular structure during use of the thrombectomy system 534. For example, the thrombectomy system 534 may include one or more conduits (e.g., the hollow cavity 104 shown in FIGS. 1A and 1B) that fluidly couple the vacuum source 544 with the catheter 502. For example, the conduits may fluidly connect the vacuum source 544 with the handle 538. The vacuum source 544 may include a manual vacuum pump, an electric vacuum pump, a diaphragm pump, a centrifugal pump, a displacement pump, a magnetically driven pump, a peristaltic pump, a wall-mounted vacuum source, or any pump configured to produce a vacuum. In an embodiment, the vacuum source 544 may include a syringe, wherein extraction of a plunger of the syringe provides the aspiration.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

What is claimed is:

1. A thrombectomy device, comprising:
a catheter including a proximal end region and a distal end region; and
at least one heatable cutter extending from the distal end region, the at least one heatable cutter configured to cut a thrombus;
wherein the thrombectomy device is switchable between a deployment state and an expanded state, wherein a maximum lateral dimension of the thrombectomy device is greater when the thrombectomy device and the at least one heatable cutters are in the expanded state than in the deployment state.

2. The thrombectomy device of claim 1, wherein the catheter includes an inner catheter and an outer catheter that is configured to move relative to the inner catheter, the at least one heatable cutter including a proximal end attached to the outer catheter and a distal end attached to the inner catheter, and wherein moving the outer catheter relative to the inner catheter switches the thrombectomy device between the deployment state and an expanded state.

3. The thrombectomy device of claim 1, further comprising an outer sheath moveably positioned over the catheter, the thrombectomy device configured to be in the deployment state when the at least one heatable cutter is disposed within the outer sheath and in the expanded state when the at least one heatable cutter is disposed outside of the outer sheath.

4. The thrombectomy device of claim 1, wherein the at least one heatable cutter includes a plurality of heatable cutters.

5. The thrombectomy device of claim 1, wherein the at least one heatable cutter includes at least one rounded, non-sharpened cutting edge.

6. The thrombectomy device of claim 1, wherein the at least one heatable cutter includes at least one sharpened cutting edge.

7. The thrombectomy device of claim 1, wherein the at least one heatable cutter includes a nickel-titanium alloy.

8. The thrombectomy device of claim 1, wherein the at least one heatable cutter includes a nickel-chromium alloy.

9. The thrombectomy device of claim 1, wherein the at least one heatable cutter includes an electrically resistive material surrounded by an insulator.

10. The thrombectomy device of claim 1, wherein the at least one heatable cutter is configured to be heated using a radio frequency alternating current.

11. The thrombectomy device of claim 1, wherein the at least one heatable cutter is configured to be heated to a maximum temperature of 130 °C.

12. A thrombectomy system, comprising:
a thrombectomy device, including:
a catheter including a proximal end region and a distal end region; and
at least one heatable cutter extending from the distal end region, the at least one heatable cutter configured to cut a thrombus;
wherein the thrombectomy device is switchable between a deployment state and an expanded state, wherein a maximum lateral dimension of the thrombectomy device is greater when the thrombectomy device and the at least one heatable cutter are in the expanded state than the deployment state; and
a stimulus source configured to provide stimuli to the at least one heatable cutter, wherein the stimuli is configured to heat the heatable cutter.

13. The thrombectomy system of claim 12, wherein the stimulus source includes an alternating current source configured to apply an alternating current exhibiting radio frequencies.

14. A method of using a thrombectomy device, the method comprising:
providing the thrombectomy device including:
a catheter including a proximal end region and a distal end region; and
at least one heatable cutter extending from the distal end region, the at least one heatable cutter configured to cut a thrombus; and
switching the thrombectomy device from a deployment state to an expanded state, wherein a maximum lateral dimension of the thrombectomy device is greater when the thrombectomy device and the at least one heatable cutter are in the expanded state than in the deployment state.

15. The method of claim 14, wherein switching the thrombectomy device from the deployment state to the expanded state includes moving at least a portion of the at least one heatable cutter away from a central, longitudinal axis of the catheter.

16. The method of claim 15, wherein:
the catheter includes an inner catheter and an outer catheter that is configured to move relative to the inner catheter, the at least one heatable cutter including a proximal end attached to the outer catheter and a distal end attached to the inner catheter; and
switching the thrombectomy device from the deployment state to the expanded state includes moving the outer catheter relative to the inner catheter.

17. The method of claim 14, further comprising heating the at least one heating cutter.

18. The method of claim 17, further comprising, after heating the at least one heatable cutter, rotating the at least one heatable cutter while moving the at least one heatable cutter into a thrombus.

19. The method of claim 17, wherein heating the at least one heatable cutter includes flowing a radio frequency alternating current through the at least one heatable cutter.

20. The method of claim 17, wherein heating the at least one heatable cutter includes heating the at least one heatable cutter to a temperature of about 90 °C to about 130 °C.
